(19) United States
(12) Patent Application Publication
(10) Pub. No.: US 2014/0315327 A1
(43) Pub. Date: Oct. 23, 2014

(54) Methods for Measuring Concentrations of Biomolecules

(71) Applicant: C2N Diagnostics, Saint Louis, MO (US)

(72) Inventors: Tim West, Saint Louis, MO (US); Andrew Corey Paoletti, Saint Louis, MO (US)

(21) Appl. No.: 14/362,105

(22) PCT Filed: Nov. 29, 2012
(86) PCT No.: PCT/US2012/067110

§ 371 Date: May 30, 2014

Related U.S. Application Data
(60) Provisional application No. 61/566,289, filed on Dec. 2, 2011.

(51) Int. Cl. G01N 33/53

(52) U.S. Cl. CPC G01N 33/5308
USPC 436/501

(57) ABSTRACT

The present invention provides methods for measuring the absolute concentration of a biomolecule of interest in a subject. Such biomolecules may be implicated in one or more neurological and neurodegenerative diseases or disorders. Also provided is a method for determining whether a therapeutic agent affects the in vivo metabolism of a central nervous system derived biomolecule. Also provided are kits for performing the methods of the invention.

### BACKGROUND OF THE INVENTION

1. Field of the Invention

The invention generally relates to methods for the diagnosis and treatment of neurological and neurodegenerative diseases, disorders, and associated processes.

2. Background Information

Alzheimer's Disease (AD) is the most common cause of dementia and is an increasing public health problem. It is currently estimated to afflict 5 million people in the United States, with an expected increase to 13 million by the year 2050 (Herbert et al, 2001, Alzheimer Dis. Assoc. Disord. 15(4): 169-173). AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup of this protein in form of amyloid plaques the brains of those with the disease. In addition the protein Tau builds up in the brain in the form of Tau tangles. AD leads to loss of memory, cognitive function, and ultimately independence and death. The disease takes a heavy personal and financial toll on the patient, the family, and society. Because of the severity and increasing prevalence of this disease in the population, it is urgent that better treatments be developed.

Currently, there are some medications that modify symptoms, however, there are no disease-modifying treatments. Disease-modifying treatments will likely be most effective when given before the onset of irreversible brain damage. However, by the time clinical diagnosis of AD is made, extensive neuronal loss has already occurred (Price et al. 2001, Arch. Neurol. 58(9): 1395-1402). Therefore, a way to identify those at risk of developing AD would be most helpful in preventing or delaying the onset of AD. Currently, there are no means of identifying the pathophysiologic changes that occur in AD before the onset of clinical symptoms or of effectively measuring the effects of treatments that may prevent the onset or slow the progression of the disease.

A need therefore exists for a sensitive, accurate, and reproducible method for quantifying biomolecules in a subject. Previous technologies used for absolute quantitation include enzyme linked immunosorbent assays (ELISAs), which use antibodies to capture and measure the concentrations. However, ELISAs quantitate total concentration or rely on isoform specific antibodies for quantitation and can, for the most part, be used to measure the concentration of only one species per assay. Antibodies used for ELISA assays must be highly specific for the protein species and the conformations of the proteins they bind and the reliance upon two antibodies binding to the protein of interest can lead to high inter- and intra-assay variability in the reported concentrations from ELISA assays. As such, a method is needed for measuring the absolute quantitation of the concentrations of one or more biomolecules in biological fluids and tissues in vivo, where the biomolecules are associated with the diagnosis and/or progression of diseases.

### SUMMARY OF THE INVENTION

Among the various aspects of the present invention is the provision of a method for calculating the concentration of one or more biomolecules in a subject. The method includes contacting a sample from the subject with a Quantitation Standard, where the Quantitation Standard is a known concentration of a labeled biomolecule of interest. The Quantitation Standard can be contacting the sample from the subject either before isolation of the biomolecules of interest from the sample or after isolation of the biomolecule from the sample. The method further includes isolating the biomolecule of interest from the sample and determining a ratio of labeled to unlabeled biomolecules in the sample, which is thereby used to calculate the concentration of the unlabeled biomolecule in the sample. In one embodiment, the method further includes normalizing the calculated concentration to a standard curve, wherein the standard curve is generated by determining two or more ratios of unlabeled biomolecules to Quantitation Standard, where the concentration of the unlabeled biomolecule is known.

In another aspect, the present invention provides an in vivo method of quantifying the concentration of one or more biomolecules in a subject. The method includes administering one or more labeled amino acids to the subject, where the labeled amino acids incorporate into a biomolecule of interest in the subject. The method further includes obtaining a sample of biological fluid or tissue from the subject, where the sample includes a labeled biomolecule fraction and an unlabeled biomolecule fraction. The sample is then contacted with a Quantitation Standard, where the Quantitation Standard includes a known concentration of a biomolecule labeled with a moiety that has a molecular weight that differs from the one or more labeled amino acids administered to the subject. The ratio of labeled biomolecule to the Quantitation Standard and the ratio of unlabeled biomolecule to the Quantitation Standard can then be used to calculate the concentrations of both labeled and unlabeled biomolecules, respectively. In one embodiment, calculating the concentration of the unlabeled biomolecule comprises multiplying the concentration of the Quantitation Standard with the determined ratio of unlabeled biomolecule to the Quantitation Standard. In another embodiment, calculating the concentration of the labeled biomolecule comprises multiplying the concentration of the Quantitation Standard with the determined ratio of labeled biomolecule to the Quantitation Standard. In yet another embodiment, the calculated concentrations of unlabeled and labeled biomolecules are normalized to each their individual standard curves, wherein the standard curve is generated by determining two or more ratios of unlabeled and labeled biomolecules to Quantitation Standard, where the concentration of unlabeled and labeled biomolecule is known.

In another aspect, the invention provides a method for measuring the in vivo metabolism of one or more biomolecules produced in the central nervous system of a subject. The method comprises administering a labeled moiety to the subject, wherein the labeled moiety is capable of crossing the blood brain barrier and incorporating into the biomolecule(s) as the one or more biomolecules is produced in the central nervous system of the subject. The method further comprises obtaining a central nervous system sample from the subject, wherein the central nervous system sample is a central nervous system tissue or fluid. The central nervous system sample comprises a labeled biomolecule fraction in which the labeled moiety is incorporated into the one or more biomolecules, and an unlabeled biomolecule fraction in which the labeled moiety is not incorporated into the one or more biomolecules. The final step of the process comprises detecting the amount of labeled biomolecule and the amount of unlabeled biomolecule for each of the one or more biomolecules, wherein the ratio of labeled biomolecule to unlabeled biomolecule for each biomolecule is directly proportional to the metabolism of said biomolecule in the subject.

In another aspect, the invention provides a method for determining whether a therapeutic agent affects the metabolism of a biomolecule produced in the central nervous system of a subject. The method comprises administering a therapeutic agent and a labeled moiety to the subject, wherein the labeled moiety is capable of crossing the blood brain barrier and incorporating into the biomolecule as it is being is produced in the central nervous system of the subject. The method further comprises obtaining a biological sample from the subject, wherein the biological sample comprises a labeled biomolecule fraction in which the labeled moiety is incorporated into the biomolecule, and an unlabeled biomolecule fraction in which the labeled moiety is not incorporated into the biomolecule. The next step of the process comprises detecting the amount of labeled biomolecule and the amount of unlabeled biomolecule, wherein the ratio of labeled biomolecule to unlabeled biomolecule is directly proportional to the metabolism of the biomolecule in the subject. The final step of the process comprises comparing the metabolism of the biomolecule in the subject to a suitable control value, wherein a change from the control value indicates the therapeutic agent affects the metabolism of the biomolecule in the central nervous system of the subject.

In another aspect, the invention provides a kit for performing the methods of the invention. In one embodiment, a kit is provided for diagnosing and/or monitoring the progression or treatment of a neurological or neurodegenerative disease in a subject. The kit includes one or more labeled moieties (e.g., labeled amino acids) and a means for administering the one or more amino acids to the subject. The kit may further include a means for obtaining a biological sample at regular time intervals from the subject. In certain embodiments, the kit will also include instructions for detecting and determining the ratio of labeled to unlabeled biomolecules of interest over time and for calculating the concentration of the unlabeled biomolecule. In one embodiment, the instructions will disclose methods for comparing the calculated concentration to certain standards and/or controls as disclosed herein.

In all aspects, the labeled moiety includes a non-radioactive isotope that is selected from the group consisting of ²H, ¹³C, ¹⁵N, ¹⁷O, ¹⁸O, ³³S, ³⁴S and ³⁶S. In one embodiment, the labeled moiety is a labeled amino acid, which can be essential or nonessential. Exemplary amino acids include, but are not limited to threonine, glutamic acid, leucine, isoleucine, and phenylalanine. Thus, in one embodiment, the labeled moiety is ¹³Cₓ-threonine, where x=1 to 4. In another embodiment, the labeled moiety is a ¹⁵N-labeled amino acid. In another embodiment, the labeled moiety is a ¹³Cₓ-labeled leucine, where x=1 to 6. In another embodiment, the labeled moiety is a ¹³Cₓ-labeled glutamic acid, where x=1 to 5. In another embodiment, the labeled moiety is a ¹³Cₓ-labeled phenylalanine, where x=1 to 9. In another embodiment, the labeled moiety is a ¹³Cₓ-labeled isoleucine, where x=1 to 6. In another embodiment, the labeled moiety is a ¹³Cₓ-labeled isoleucine and a ¹³C_{y}-labeled phenylalanine, where x=1 to 6, and y=1 to 9.

In all aspects, the biomolecule may be a peptide, lipid, nucleic acid, or carbohydrate. In one embodiment, the biomolecule is a peptide that is synthesized in the central nervous system (CNS) such as Tau, amyloid-beta (Aβ), alpha-synuclein, apolipoprotein E, apolipoprotein J, amyloid precursor protein (APP), alpha-2-macroglobulin, S100B, myelin basic protein, TDP-43, superoxide dismutase-1, huntingtin, an interleukin, and TNF. In aspects of the invention where two or more biomolecules are assayed, the biomolecules may be isoforms of the same protein. As such, in one embodiment, the biomolecule may be one or more of Tau-4R2N, Tau-4R1N, Tau-4R0N, Tau-3R2N, Tau-3R1N, Tau-3R0N.

Other aspects and features of the invention are described in more detail below.

### BRIEF DESCRIPTION OF THE DRAWINGS

FIG. 1 shows the ratio of ¹³C₆ labeled Tau to unlabeled Tau as isolated from cell culture media after adding ¹³C₆ leucine to the media. This demonstrates the metabolic incorporation of ¹³C₆ leucine into Tau as it is being produced by the cells.

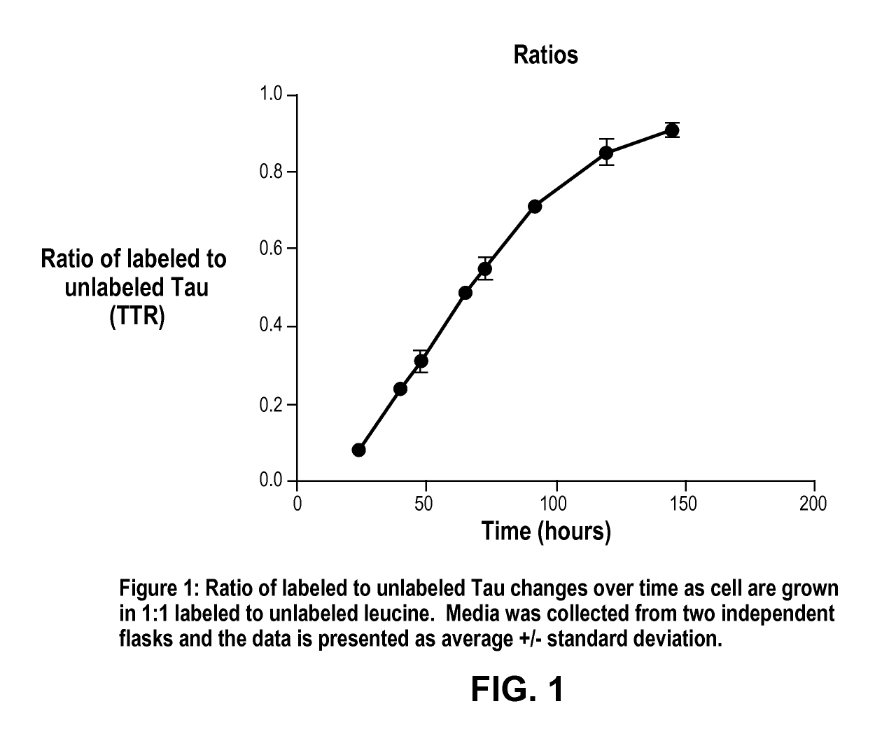

FIG. 2 shows a standard curve of for SISAQ-Tau. The curve is linear in the range of 5 ng/mL to 51 pg/mL (2.5 fold dilutions). This curve was used to measure the concentration of Tau in two CSF samples (run in triplicate).

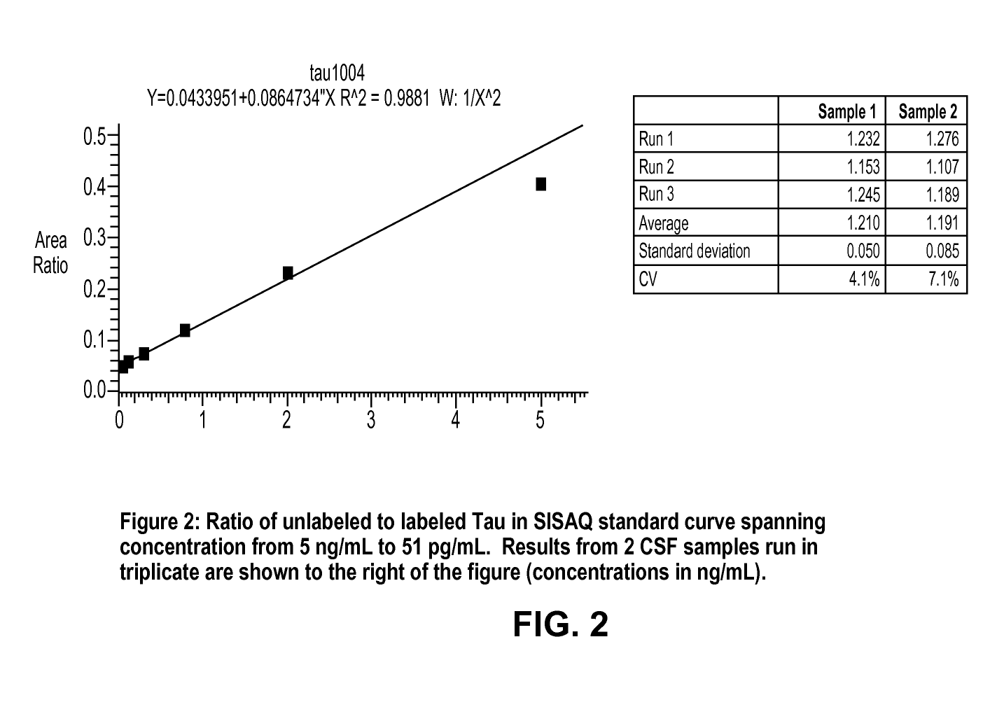

FIG. 3 shows a chromatogram for 7 Tau derived peptides based on LysN digestion. The table shows the peptides along with the m/z of the parent ion.

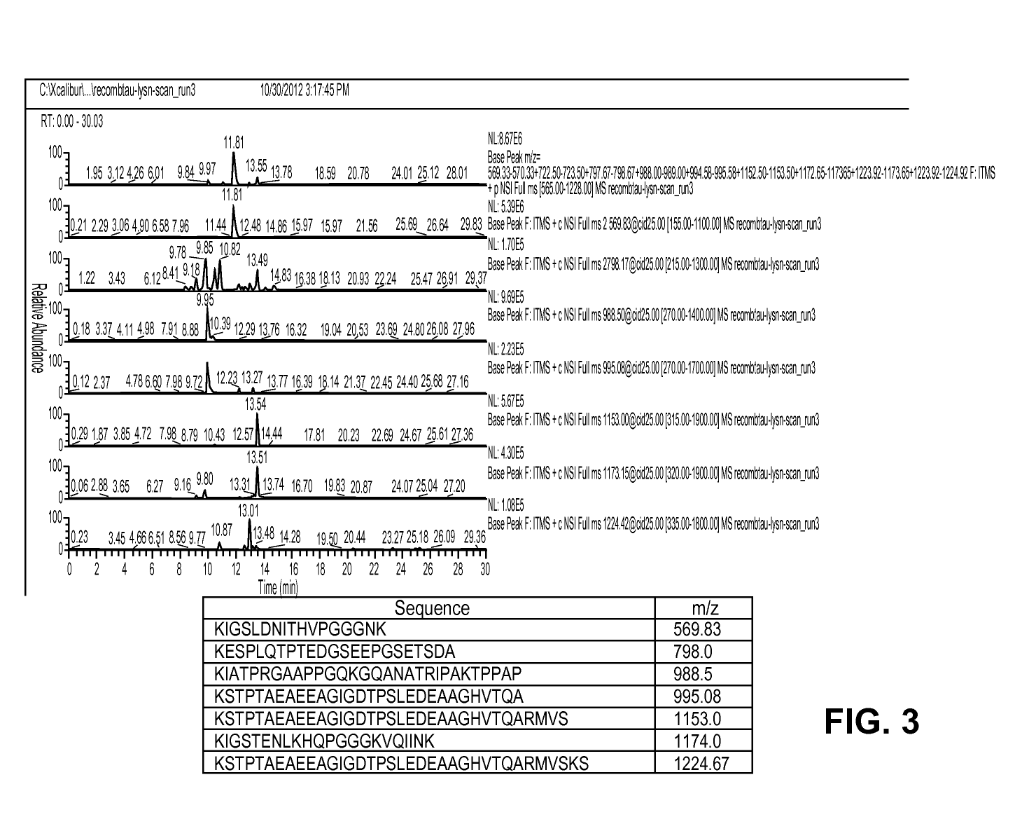

FIG. 4 shows a spectrum from a LysN digest of Tau. The peptide is phosphorylated on Threonine 40 (SEQ ID NO:12).

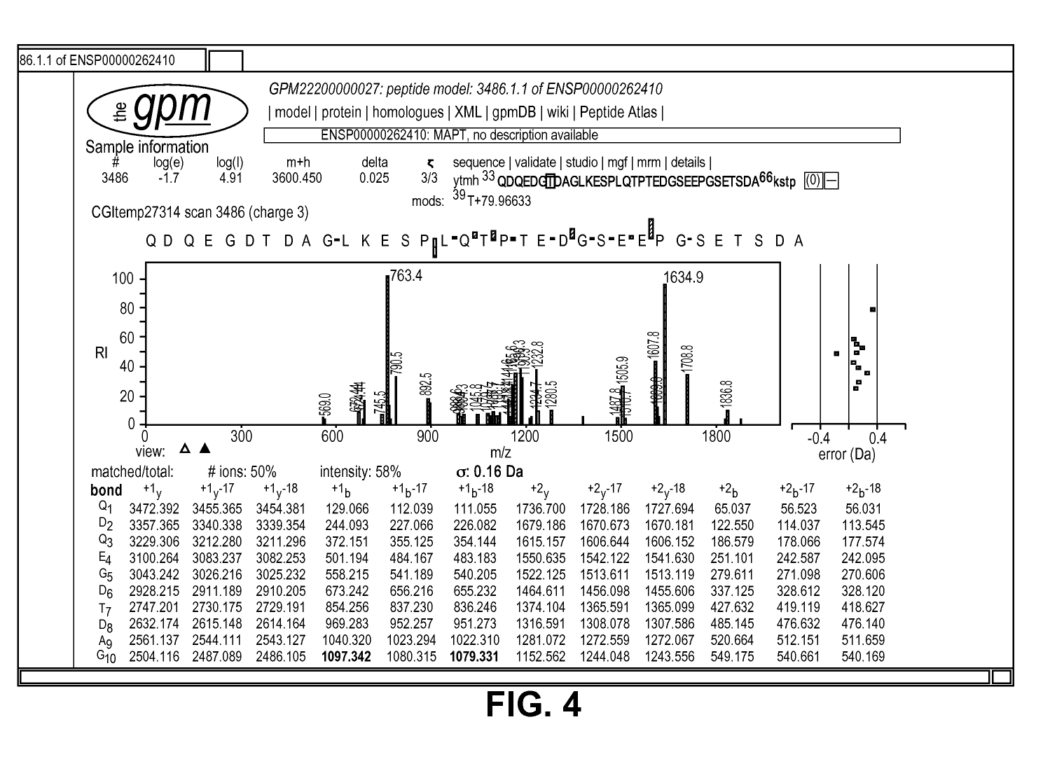

### DETAILED DESCRIPTION OF THE INVENTION

The present invention is based, in part, on the discovery that stable isotope labeling of biomolecules leads to small differences in molecular weight of the biomolecules, but does not alter the physical or chemical properties of the biomolecules. Using the techniques provided herein, analysis of biomolecules can be used to diagnose and/or treat a subject having or at risk of developing a neurological or neurodegenerative disorder. Accordingly, the present invention provides methods and kits useful for calculating the concentration of one or more biomolecules of interest in a subject.

The invention also provides a method to assess whether a therapeutic agent affects the production or clearance rate of biomolecules in the subject, where the biomolecules are relevant to neurological or neurodegenerative diseases. Accordingly, the method may be used to determine the optimal doses and/or optimal dosing regimes of the therapeutic agent. Additionally, the method may be used to determine which subjects respond better to a particular therapeutic agent. For example, subjects with increased production of the biomolecule may respond better to one therapeutic agent, whereas subjects with decreased clearance of the biomolecule may respond better to another therapeutic agent. Alternatively, subjects with one particular genotype may respond better to a particular therapeutic agent than those with a different genotype. Finally, by allowing isoform specific quantitation, the method may be used to determine whether a therapeutic agent can modulate the production of a biomolecule by switching production of one isoform to another isoform of the same biomolecule.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. In addition, the term “subject” may refer to a culture of cells, where the methods of the invention are performed in vitro to assess, for example, efficacy of a therapeutic agent.

As used herein, the terms “sample” and “biological sample” refer to any sample suitable for the methods provided by the present invention. A sample of cells used in the present method can be obtained from tissue samples or bodily fluid from a subject, or tissue obtained by a biopsy procedure (e.g., a needle biopsy) or a surgical procedure. In certain embodiments, the biological sample of the present invention is a sample of bodily fluid, e.g., cerebral spinal fluid (CSF), blood, plasma, urine, saliva, and tears.

As disclosed herein, stable isotope labeling of biomolecules leads to small differences in molecular weight of the biomolecules, but does not alter the physical or chemical properties of the biomolecules. Thus, the biomolecules will bind to antibodies and elute off a liquid chromatography column in an identical fashion. Sensitive instruments, such as mass spectrometers, provide the ability to measure small differences in weight between labeled and unlabeled biomolecules.

Accordingly, in one aspect, the invention provides a method of calculating the concentration of a biomolecule in a subject. In one embodiment, the method includes contacting a sample from the subject with a Quantitation Standard. As used herein, a “Quantitation Standard” refers to a known concentration of a labeled biomolecule, which has a distinct molecular weight from other labeled or unlabeled biomolecules that may exist in the sample. Thereafter, a sensitive measuring device, such as a mass spectrometer, a tandem mass spectrometer, or a combination of both, is used to measure the ratio of labeled to unlabeled biomolecules. Since the physical properties of the labeled and unlabeled biomolecules are identical, the ratio measured by the mass spectrometer is identical to the ratio in the original sample. Thus, by adding a known amount of one or more biomolecules, each labeled with a unique isotopic label, the invention provides the ability to quantitate the amount of those biomolecules that have different isotopic composition.

As used herein, the term “biomolecule” refers to any organic molecule in a living organism. Exemplary biomolecules include, but are not limited to, peptides, lipids, nucleic acids, and carbohydrates. In one embodiment, the biomolecule is a peptide, such as a protein, that is synthesized in the central nervous system (CNS) of the subject. Exemplary proteins that can be measured by the methods of the invention include, but are not limited to, Tau (associated with Alzheimer's Disease), amyloid-β (Aβ) and its variants, soluble amyloid precursor protein (APP), apolipoprotein E (isoforms 2, 3, or 4), apolipoprotein J (also called clusterin), phospho Tau, glial fibrillary acidic protein, alpha-2 macroglobulin, alpha-synuclein, S100B, myelin basic protein (implicated in multiple sclerosis), prions, interleukins, TDP-43, superoxide dismutase-1, huntingtin, tumor necrosis factor (TNF), heat shock protein 90 (HSP90), and combinations thereof. Additional biomolecules that may be targeted include products of, or proteins or peptides that interact with, GABAergic neurons, noradrenergic neurons, histaminergic neurons, seratonergic neurons, dopaminergic neurons, cholinergic neurons, and glutaminergic neurons. In one embodiment, the protein whose in vivo concentration is measured may be an apolipoprotein E protein. In another embodiment, the protein whose in vivo concentration is measured may be alpha-synuclein. In another embodiment, the protein whose in vivo concentration is measured may be Aβ or its variants or isoforms. In another embodiment, the protein whose in vivo concentration is measured may be Tau or its variants or isoforms. Exemplary isoforms of Tau whose concentrations may be measured include, but are not limited to, the following phosphorylated or unphosphorylated isoforms of Tau: Tau-4R2N, Tau-4R1N, Tau-4R0N, Tau-3R2N, Tau-3R1N, Tau-3R0N.

By way of example and not limitation, it is noted that several unique isoforms of Tau exist in CSF, and that these isoforms can be post-translationally modified in several ways including phosphorylation. Trypsin digestion of Tau yields several peptides (see Table 1). Thus, quantitation of some of these peptides allows for calculation of the concentration of these isoforms in the original biological fluid.

TABLE 1
Tryptic peptides of Tau identified by mass spectrometer.

| Peptide sequence | m/z | Isoform present | Sequence ID |
|---|---|---|---|
| IGSLDNITHVPGGGNK | 790.25 | All | SEQ ID NO: 1 |
| SGYSSPGSPGTPGSR | 697.4 | All | SEQ ID NO: 2 |
| STPTAEAEEAGIGDTPSLEDEAAGHVTQAR | 1004.6 | 1N isoforms only | SEQ ID NO: 3 |
| | | (412/381) | |
| KESPLQTPTEDGSEEPGSETSDA | 196 | All except for 0N | SEQ ID NO: 4 |
| | | (383/352) | |

As such, the methods provide the ability to measure concentrations of various isoforms of Tau, such as fragments produced after digestion with an endoprotease (e.g., trypsin, LysN, or V8 protease). Exemplary fragments of Tau isoforms include, but are not limited to regions of Tau that are different between the different isoforms and their boundaries, such as the N-terminal region (2N/1N/0N) and the C-terminal repeat region (4R/3R).

Exemplary peptides of Tau following LysN digestion include the following:

TABLE 2
LysN peptides of Tau

| | | SEQ ID |
|---|---|---|
| Sequence | m/z | NO: |
| KIGSLDNITHVPGGGNK | 569.83 | 5 |
| KESPLQTPTEDGSEEPGSETSDA | 798.0 | 6 |
| KIATPRGAAPPGQKGQANATRIPAKTPPAP | 988.5 | 7 |
| KSTPTAEAEEAGIGDTPSLEDEAAGHVTQA | 995.08 | 8 |
| KSTPTAEAEEAGIGDTPSLEDEAAGHVTQA | 1153.0 | 9 |

RMVS

| | | |
|---|---|---|
| KIGSTENLKHQPGGGKVQIINK | 1174.0 | 10 |
| KSTPTAEAEEAGIGDTPSLEDEAAGHVT | 1224.67 | 11 |

QARMVSKS

As used herein, the term “nucleic acid” refers to DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule.

The terms “polypeptide,” “peptide” and “protein” are used interchangeably herein to refer to two or more amino acid residues joined to each other by peptide bonds or modified peptide bonds, i.e., peptide isosteres. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polyeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

Several different moieties may be used to label the biomolecule of interest. Generally speaking, the two types of labeling moieties utilized in the method of the invention are radioactive isotopes and non-radioactive (stable) isotopes. In one embodiment, non-radioactive isotopes may be used and measured by mass spectrometry. Preferred stable isotopes include deuterium (²H), ¹³C, ¹⁵N, ^{17 or 18}O and ^{33, 34, or 36}S, but it is recognized that a number of other stable isotopes that change the mass of an atom by more or less neutrons than is seen in the prevalent native form would also be effective. A suitable label generally will change the mass of the biomolecule under study such that it can be detected in a mass spectrometer. In one embodiment, the biomolecule to be measured may be a peptide or protein, and the labeled moiety may be an amino acid comprising a non-radioactive isotope (e.g., ¹³C). In another embodiment, the biomolecule to be measured may be a nucleic acid, and the labeled moiety may be a nucleoside triphosphate comprising a non-radioactive isotope (e.g., ¹⁵N). Alternatively, a radioactive isotope may be used, and the labeled biomolecules may be measured with a scintillation counter (or via nuclear scintigraphy) as well as by a mass spectrometer. One or more labeled moieties may be used simultaneously or in sequence.

Thus, in one embodiment, when the method is employed to measure the concentration of proteins, the labeled moiety typically will be an amino acid. Those of skill in the art will appreciate that several amino acids may be used to provide the label of biomolecules. Generally, the choice of amino acid is based on a variety of factors such as: (1) The amino acid generally is present in at least one residue of the protein or peptide of interest. (2) The amino acid is generally able to reach the site of protein production and rapidly equilibrate tissue or cellular barriers. And (3) commercial availability of the desired amino acid (i.e., some amino acids are much more expensive or harder to manufacture than others).

In one embodiment, the amino acid is an essential amino acid (not produced by the body), so that a higher percent of labeling may be achieved. In another embodiment, the amino acid is a nonessential amino acid. Exemplary amino acids include, but are not limited to threonine, glutamic acid, leucine, isoleucine, and phenylalanine. As such, in one embodiment, the labeled moiety is ¹³Cₓ-threonine, where x=1 to 4. In another embodiment, the labeled amino acid is one or more of a ¹⁵N-labeled amino acid, a ¹³Cₓ-labeled glutamic acid, where x=1 to 5, a ¹³Cₓ-labeled leucine, where x=1 to 6, a ¹³Cₓ-labeled phenylalanine, where x=1 to 9, a ¹³Cₓ-labeled isoleucine, where x=1 to 6, a ¹³Cₓ-labeled isoleucine and a ¹³C_{y}-labeled phenylalanine, where x=1 to 6, and y=1 to 9. For example, ¹³C₆-phenylalanine, which contains six ¹³C atoms, may be used to label a biomolecule of interest (e.g., a CNS derived protein). In yet another embodiment, ¹³C₆-leucine is used to label Aβ or Tau.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

The labeled moiety (e.g., labeled amino acid) may be administered to a subject by several methods. Suitable routes of administration include intravenously, intra-arterially, subcutaneously, intraperitoneally, intramuscularly, or orally. In one embodiment, the labeled moiety may be administered by intravenous infusion. In another embodiment, the labeled moiety may be orally ingested.

The labeled moiety may be administered slowly over a period of time, as a large single dose depending upon the type of analysis chosen (e.g., steady state or bolus/chase), or slowly over a period of time after an initial bolus dose. To achieve steady-state levels of the labeled biomolecule, the labeling time generally should be of sufficient duration so that the labeled biomolecule may be reliably quantified. In one embodiment, the labeled moiety is administered as a single oral dose. In another embodiment, the labeled moiety is administered for a period of time ranging from about one hour to about 36 hours. In another embodiment, the labeled moiety is administered for a period of time ranging from about 6 hours to about 12 hours. In yet another embodiment, the labeled moiety is administered for a period of time ranging from about 9 hours to about 12 hours. In yet another embodiment, the labeled moiety is administered for a period of time ranging from about 9 hours to about 24 hours.

The rate of administration of the labeled moiety may range from about 0.5 mg/kg/hr to about 5 mg/kg/hr. In one embodiment, the rate of administration of labeled leucine is from about 1 mg/kg/hr to about 3 mg/kg/hr. In another embodiment, the rate of administration of labeled leucine is from 1.8 mg/kg/hr to about 2.5 mg/kg/hr. In another embodiment, the labeled leucine may be administered as a bolus of between about 50 and about 500 mg/kg body weight of the subject, between about 50 and about 300 mg/kg body weight of the subject, or between about 100 and about 300 mg/kg body weight of the subject. In yet another embodiment, the labeled leucine may be administered as a bolus of about 200 mg/kg body weight of the subject. In an alternate embodiment, the labeled leucine may be administered intravenously as detailed above after an initial bolus of between about 0.5 to about 10 mg/kg, between about 1 to about 4 mg/kg, or about 2 mg/kg body weight of the subject.

Those of skill in the art will appreciate that the amount (or dose) of the labeled moiety can and will vary. Generally, the amount is dependent on (and estimated by) the following factors: (1) The type of analysis desired. For example, to achieve a steady state of about 15% labeled leucine in plasma requires about 2 mg/kg/hr over about 9 hr after an initial bolus of 2 mg/kg over 10 min. In contrast, if no steady state is required, a large bolus of labeled moiety (e.g., 1 or 5 grams of labeled leucine) may be given initially. (2) The protein under analysis. For example, if the protein is being produced rapidly, then less labeling time may be needed and less label may be needed—perhaps as little as 0.5 mg/kg over 1 hour. However, most proteins have half-lives of hours to days and, so more likely, a continuous infusion for 4, 9 or 12 hours may be used at 0.5 mg/kg to 4 mg/kg. And (3) the sensitivity of detection of the label. For example, as the sensitivity of label detection increases, the amount of label that is needed may decrease.

It should be understood that more than one labeled moiety may be used in a single subject. This would allow multiple labeling of the same biomolecule and may provide information on the production or clearance of that biomolecule at different times. For example, a first label may be given to subject over an initial time period, followed by a pharmacologic agent (drug), and then a second label may be administered. In general, analysis of the samples obtained from the subject would provide a measurement of concentrations of biomolecules of interest before AND after drug administration, directly measuring the pharmacodynamic effect of the drug in the same subject. Alternatively, multiple labels may be used at the same time to increase labeling of the biomolecule.

Thus, once disease is established and a treatment protocol is initiated, the methods of the invention may be repeated on a regular basis to monitor the concentration(s) of biomolecule(s) of interest in the subject. The results obtained from successive assays may be used to show the efficacy of treatment over a period ranging from several days to months. Accordingly, another aspect of the invention is directed to methods for monitoring a therapeutic regimen for treating a subject having a neurological or neurodegenerative disorder. A comparison of the concentration(s) of biomolecule(s) of interest prior to and during therapy will be indicative of the efficacy of the therapy. Therefore, one skilled in the art will be able to recognize and adjust the therapeutic approach as needed.

The method of the invention provides that a sample be obtained from the subject such that the in vivo concentration of one or more biomolecules of interest can be determined. In one embodiment, the sample is a body fluid. Suitable body fluids include, but are not limited to, cerebral spinal fluid (CSF), blood plasma, blood serum, urine, saliva, perspiration, and tears. It should be understood that biological fluids typically contain a multitude of quantifiable biomolecules. For example, where the sample is CSF, exemplary biomolecules that can be quantified include, but are not limited to, Tau, variants of Tau, amyloid-beta protein, variants of amyloid-beta protein (Aβ), digestion products of amyloid-beta protein, amyloid precursor protein (APP), apolipoprotein E, apolipoprotein J, alpha-synuclein, or any combination thereof. In another embodiment, the sample is a tissue sample, such as a sample of tissue from the central nervous system (CNS). The sample generally will be collected using standard procedures well known to those of skill in the art.

In one embodiment, the sample is a CNS sample, which includes, but is not limited to, tissue from the central nervous system, which comprises brain tissue and spinal cord tissue. In one embodiment of the invention, the CNS sample may be taken from brain tissue, including, but not limited to, tissue from the forebrain (e.g., cerebral cortex, basal ganglia, hippocampus), the interbrain (e.g., thalamus, hypothalamus, subthalamus), the midbrain (e.g., tectum, tegmentum), or the hindbrain (e.g., pons, cerebellum, medulla oblongata). In another embodiment, the CNS sample may be collected from spinal cord tissue. In still other embodiments, CNS samples from more than one CNS region may be taken. Accordingly, the concentration of a biomolecule of interest may be measured in different CNS samples, e.g., in the cortex and the hippocampus, simultaneously.

CNS samples may be obtained by known techniques. For instance, brain tissue or spinal cord tissue may be obtained via dissection or resection. Alternatively, CNS samples may be obtained using laser microdissection. The subject may or may not have to be sacrificed to obtain the sample, depending on the CNS sample desired and the subject utilized.

In general when the biomolecule under study is a peptide or protein, the invention provides that a first sample may be taken from a subject prior to administration of the labeled moiety to provide a baseline. After administration of the labeled moiety (e.g., labeled amino acid), one or more samples are obtained from the subject. As will be appreciated by those of skill in the art, the number of samples and when the samples are taken generally will depend upon a number of factors such as: the type of analysis, type of administration, the protein of interest, the rate of metabolism, the type of detection, and the type of subject.

In one embodiment, the sample is obtained from the subject at a single predetermined time point, for example, within an hour of labeling. In general, for proteins with fast metabolism, samples obtained during the first 12-18 hours after the start of administration of the labeled moiety may be used to determine the rate of production of the biomolecule of interest, and samples taken during 24-36 hrs after the start of administration of the labeled moiety may be used to determine the clearance rate of the biomolecule of interest. In another embodiment, the sample is obtained from the subject hourly from 0 to 12 hours, 0 to 24 hours, or 0 to 36 hours. In yet another embodiment, samples may be taken from an hour to days or even weeks apart depending upon the production and clearance rates of the biomolecule of interest.

It should be understood that if samples at different time-points are desired, more than one subject may be used. For instance, one subject may be used for a baseline sample, another subject for a time-point of one hour post administration of the labeled moiety, another subject for a time-point six hours post administration of the labeled moiety.

Accordingly, the present invention provides that detection of the amount of labeled biomolecule and the amount of unlabeled biomolecule in the sample may be used to determine the ratio of labeled biomolecule to unlabeled biomolecule, which in turn, may be used to calculate the concentration of the biomolecule of interest in the subject. In one embodiment, the ratio is determined by means of detecting changes in mass of the labeled biomolecule (e.g., peptide or protein) with respect to the unlabeled biomolecule. Exemplary means for detecting differences in mass between the labeled and unlabeled biomolecules include, but are not limited to, liquid chromatography mass spectrometry, gas chromatography mass spectrometry, MALDI-TOF mass spectrometry, and tandem mass spectrometry.

However, prior to detecting the ratio of labeled biomolecule to unlabeled biomolecule, it may be desirable to isolate and/or separate the biomolecule of interest from other biomolecules in the sample. Thus, in one embodiment, immunoprecipitation may be used to isolate and purify the biomolecule (e.g., peptide or protein) of interest before it is analyzed. In another embodiment, the biomolecule of interest may be isolated or purified by affinity chromatography or immunoaffinity chromatography. Alternatively, mass spectrometers having chromatography setups may be used to separate biomolecules without immunoprecipitation, and then the biomolecule of interest may be measured directly. In an exemplary embodiment, the protein of interest may be immunoprecipitated and then analyzed by a liquid chromatography system interfaced with a tandem MS unit equipped with an electrospray ionization source (LC-ESI-tandem MS).

In another aspect, the invention provides that multiple biomolecules in the same sample may be measured simultaneously. That is, both the amount of unlabeled and labeled biomolecule may be detected and measured separately or at the same time for multiple biomolecules. As such, the invention provides a useful method for screening changes in concentration, and production and clearance of one or more biomolecules on a large scale (i.e., proteomics/metabolomics) and provides a sensitive means to detect and measure biomolecules involved in the underlying pathophysiology. In aspect, the invention also provides a means to measure multiple types of biomolecules. In this context, for example, a protein and a lipid may be measured simultaneously or sequentially.

Once the amount of labeled and unlabeled biomolecule has been detected in a sample, the ratio or percent of labeled biomolecule to unlabeled biomolecule may be determined. Thereafter, the concentration of the unlabeled biomolecule in the sample can be determined. In other words, since a known amount of labeled biomolecule is added to an unknown amount of biomolecules and the ratio of labeled to unlabeled is measured, the concentration of the unlabeled biomolecules can be calculated from the ratio as follows:

Concentration of unlabeled=(ratio of unlabeled to labeled)×(concentration of labeled). (i)

The equation may be simplified as:

Concentration of unlabeled=(ratio of unlabeled:Quantitation Standard)×(concentration of Quantitation Standard). (ii)

Conversely, if a known amount of unlabeled is added to an unknown amount labeled the concentration of the labeled can be calculated as follows:

Concentration of labeled=(ratio of labeled to unlabeled)×(concentration of unlabeled). (iii)

In addition, if a known amount of biomolecule 1, labeled with label 1, is added to an unknown amount of biomolecule 2, labeled with label 2, the concentration of the biomolecule 2 can be calculated as follows:

Concentration of label 2=(ratio of label 2 to label 1)×(concentration of label 1). (iv)

Similarly, if a known amount of biomolecule 1, labeled with label 1, is added to an unknown amount of biomolecule 2, labeled with label 2, and biomolecule 3, labeled with label 3, the concentration of the biomolecule 2 and biomolecule 3 can be calculated as follows:

Concentration of label 2=(ratio of label 2 to label 1)×(concentration of label 1) (v)

Concentration of label 3=(ratio of label 3 to label 1)×(concentration of label 1). (vi)

Finally, if a known amount of biomolecule 1, labeled with label 1, is added to an unknown amount of biomolecule 2, labeled with label 2, and an unknown amount of unlabeled biomolecule 3, the concentration of the biomolecule 2 and unlabeled biomolecule can be calculated as follows:

Concentration of label 2=(ratio of label 2 to label 1)×(concentration of label 1) (vii)

Concentration of unlabeled=(ratio of unlabeled to label 1)×(concentration of label 1). (viii)

In another embodiment, the methods further include the step of normalizing the calculated concentration to a standard curve based on the curve fitting equation generated by the standard curve. The standard curve used herein is generated by determining two or more ratios of unlabeled biomolecules to their respective Quantitation Standards, where the concentration of the unlabeled biomolecule of interest is known.

In another aspect, the invention allows measurement of the labeled and unlabeled protein at the same time, so that the ratio of labeled to unlabeled protein, as well as other calculations, may be made. Those of skill in the art will be familiar with the first order kinetic models of labeling that may be used with the method of the invention. For example, the fractional synthesis rate (FSR) may be calculated. The FSR equals the initial rate of increase of labeled to unlabeled protein divided by the precursor enrichment. Likewise, the fractional clearance rate (FCR) may be calculated. In addition, other parameters, such as lag time and isotopic tracer steady state, may be determined and used as measurements of the protein's metabolism and physiology. Also, modeling may be performed on the data to fit multiple compartment models to estimate transfer between compartments. Of course, the type of mathematical modeling chosen will depend on the individual protein synthetic and clearance parameters (e.g., one-pool, multiple pools, steady state, non-steady-state, compartmental modeling, etc.). As used herein, “steady state” refers to a state during which there is insignificant change in the measured parameter over a specified period of time.

Stable isotope kinetic labeling (SILK) methodology has been shown to detect metabolic incorporation of stable (non-radioactive) isotopes into newly synthesized proteins in the cerebrospinal fluid of living subject. For detailed information regarding SILK, see U.S. Pub. Nos. 2008/0145941 and 2009/0142766, and International PCT Pub. No. WO 2006/107814, the entire content of each of which is incorporated herein by reference). SILK makes it possible to measure the production and clearance rates of proteins in the central nervous system. Thus far, this methodology has been applied to measuring the production and clearance of the amyloid beta protein (Aβ) implicated in Alzheimer's disease (AD).

However, until now, the current version of the SILK assay measures only the metabolism of total Aβ since the assay measures incorporation of a “label” (i.e., an amino acid or biomolecule which contain atoms with a different isotopic composition than what is found in nature) into the 17-28 peptide of Aβ. Such an assay allows for the measurement of the biologic activity of Aβ production inhibitors but not any type of drugs or other compounds that modulate the metabolism of Tau. As such, while Aβ is provided as an example in this embodiment, it should be understood that the methods provided herein may apply to any protein (e.g., Tau).

Accordingly, in one aspect, Tau is isolated from the biologic samples by immunoprecipitation using an antibody that recognizes Tau. In this embodiment, the isolated peptides are eluted from the antibody, for example by using formic acid and then digested with trypsin or another protease. Contrary to the original version of the SILK-Aβ™ assay, which relies on quantitation of the 17-28 tryptic fragment of Aβ, the invention expands on the assay to measure the concentration of Tau.

The term “antibody” as used in this invention is meant to include intact molecules of polyclonal or monoclonal antibodies, as well as fragments thereof, such as Fab and F(ab′)₂, Fv and SCA fragments which are capable of binding an epitopic determinant. The term “specifically binds” or “specifically interacts,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less.

Accordingly, the production of protein is typically based upon the rate of increase of the labeled/unlabeled protein ratio over time (i.e., the slope, the exponential fit curve, or a compartmental model fit defines the rate of protein production). For these calculations, a minimum of one sample is typically required (one could estimate the baseline label), two are preferred, and multiple samples are more preferred to calculate an accurate curve of the uptake of the label into the protein (i.e., the production rate). If multiple samples are used or preferred, the samples need not be taken from the same subject. For instance, proteins may be labeled in five different subjects at time point zero, and then a single sample taken from each subject at a different time point post-labeling.

Conversely, after the administration of labeled amino acid is terminated, the rate of decrease of the ratio of labeled to unlabeled protein typically reflects the clearance rate of that protein. For these calculations, a minimum of one sample is typically required (one could estimate the baseline label), two are preferred, and multiple samples are more preferred to calculate an accurate curve of the decrease of the label from the protein over time (i.e., the clearance rate). If multiple samples are used or preferred, the samples need not be taken from the same subject. For instance, proteins may be labeled in five different subjects at time point zero, and then a single sample taken from each subject at a different time point post-labeling. The amount of labeled protein in a CNS sample at a given time reflects the production rate or the clearance rate (i.e., removal or destruction) and is usually expressed as percent per hour or the mass/time (e.g., mg/hr) of the protein in the subject.

Combined with stable isotope labeling kinetics (SILK) for measuring the ratio of labeled biomolecules at different time points after infusion with a labeled moiety, the methodology presented herein allows for the calculation of absolute concentration of newly synthesized biomolecules (e.g., peptides or proteins) and/or the absolute concentration of each of the isoforms of that biomolecule.

The method of the invention may be used to diagnose or monitor the progression of a neurological or neurodegenerative disease by measuring the in vivo concentration of one or more biomolecules of interest in a subject. Additionally, the methods of the invention may be used to monitor the treatment of a neurological or neurodegenerative disease by measuring the in vivo concentration of a biomolecule of interest in a subject. The concentration of the biomolecule may be linked to a neurological or neurodegenerative disease such that any increase or decrease may be indicative of the presence or progression of the disease. Thus, the calculated concentration of one or more biomolecules of interest may be compared to the concentration of the same biomolecules in a corresponding normal sample, to the concentration of the same biomolecules in a subject of known neurological or neurodegenerative disease state, to the concentration of the same biomolecules from the same subject determined at an earlier time, or any combination thereof.

In addition, such methods may help identify an individual as having a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the disease.

As used herein a “corresponding normal sample” refers to a sample from the same organ and/or of the same type as the sample being examined. In one aspect, the corresponding normal sample comprises a sample of cells obtained from a healthy individual. Such a corresponding normal sample can, but need not be, from an individual that is age-matched and/or of the same sex as the individual providing the sample being examined. In another aspect, the corresponding normal sample comprises a sample of cells obtained from an otherwise healthy portion of tissue of the subject from which the sample being tested is obtained.

Reference to the concentration of biomolecules in a subject of known neurological or neurodegenerative disease state includes a predetermined concentration of a biomolecule linked to a neurological or neurodegenerative disease. Thus, the concentration may be compared to a known concentration of biomolecules obtained from a sample of a single individual or may be from an established cell line of the same type as that of the subject. In one aspect, the established cell line can be one of a panel of such cell lines, wherein the panel can include different cell lines of the same type of disease and/or different cell lines of different diseases associated with the same biomolecule. Such a panel of cell lines can be useful, for example, to practice the present method when only a small number of cells can be obtained from the subject to be treated, thus providing a surrogate sample of the subject's cells, and also can be useful to include as control samples in practicing the present methods.

Exemplary neurological or neurodegenerative diseases that may be linked to the concentration ranges of biomolecules of interest include, but are not limited to, Alzheimer's Disease, Pick's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases (e.g., Creutzfeldt-Jakob Disease, bovine spongiform encephalopathy or Mad Cow Disease, and scrapie), Lewy Body Disease, schizophrenia, Amyotrophic Lateral Sclerosis (ALS or Lou Gehrig's Disease) or other motor neuron diseases, restless legs syndrome, epilepsy or other seizure disorders, tremors, depression, mania, anxiety disorders, brain trauma or injury, narcolepsy, insomnia or other sleep disorders, autism, normal pressure hydrocephalus, pain disorders or syndromes, migraines, cluster headaches or other forms of headache, spinocerebellar disorders, muscular dystrophies, myasthenia gravis, retinitis pigmentosa or other forms of retinal degeneration. It is also envisioned that the method of the invention may be used to study the normal physiology, metabolism, and function of the CNS.

In another aspect, the present invention provides a method for assessing whether a therapeutic agent used to treat a neurological or neurodegenerative disease affects the concentration of a biomolecule of interest in the subject. For example, the concentration of the biomolecule may be measured to determine if a given therapeutic agent results in an increase, or a decrease in the concentration of the biomolecule. In one embodiment, the method is performed in vivo, as herein described. In another embodiment, the method is performed in vitro utilizing a culture of cells, where the culture of cells is the “subject” in the methods described herein. Accordingly, use of the methods provided herein will allow those of skill in the art to accurately determine the degree of change in the concentration of the biomolecule of interest, and correlate these measurements with the clinical outcome of the disease modifying treatment. Results from this aspect of the invention, therefore, may help determine the optimal doses and frequency of doses of a therapeutic agent, may assist in the decision-making regarding the design of clinical trials, and may ultimately accelerate validation of effective therapeutic agents for the treatment of neurological or neurodegenerative diseases.

Thus, the method of the invention may be used to predict which subjects will respond to a particular therapeutic agent. For example, subjects with increased concentrations of a particular biomolecule may respond to a particular therapeutic agent differently than subjects with decreased concentrations of the biomolecule. In particular, results from the method may be used to select the appropriate treatment (e.g., an agent that blocks the production of the biomolecule or an agent that increases the clearance of the biomolecule) for a particular subject. Similarly, results from the method may be used to select the appropriate treatment for a subject having a particular genotype.

The method for predicting which subjects will respond to a particular therapeutic agent include administering a therapeutic agent and a labeled moiety to the subject, wherein the labeled moiety is incorporated into the biomolecule as it is produced in the subject. In one embodiment, the therapeutic agent may be administered to the subject prior to the administration of the labeled moiety. In another embodiment, the labeled moiety may be administered to the subject prior to the administration of the therapeutic agent. The period of time between the administration of each may be several minutes, an hour, several hours, or many hours. In still another embodiment, the therapeutic agent and the labeled moiety may be administered simultaneously. The method further includes collecting at least one biological sample, which includes labeled and unlabeled biomolecules, determining a ratio of the labeled biomolecule and unlabeled biomolecule in the sample, and calculating the concentration of the unlabeled biomolecule in the subject. Thereafter, a comparison of the calculated concentration to a control value will determine whether the therapeutic agent alters the concentration (e.g., by altering the rate of production or the rate of clearance) of the biomolecule in the subject.

Those of skill in the art will appreciate that the therapeutic agent can and will vary depending upon the neurological or neurodegenerative disease or disorder to be treated and/or the biomolecule whose metabolism is being analyzed. In embodiments in which the biomolecule is Tau, non-limiting examples of suitable therapeutic agents include Tau metabolism modulators, Tau kinase inhibitors, cathepsin D inhibitors, and Tau aggregation inhibitors. Other suitable AD therapeutic agents include hormones, neuroprotective agents, and cell death inhibitors. Many of the above mentioned therapeutic agents may also affect the in vivo metabolism of other proteins implicated in neurodegenerative disorders. Furthermore, therapeutic agents that may affect the in vivo metabolism of synuclein include sirtuin 2 inhibitors, synuclein aggregation inhibitors, proteosome inhibitors, etc.

The therapeutic agent may be administered to the subject in accordance with known methods. Typically, the therapeutic agent will be administered orally, but other routes of administration such as parenteral or topical may also be used. The amount of therapeutic agent that is administered to the subject can and will vary depending upon the type of agent, the subject, and the particular mode of administration. Those skilled in the art will appreciate that dosages may be determined with guidance from Goodman & Goldman's The Pharmacological Basis of Therapeutics, Tenth Edition (2001), Appendix II, pp. 475-493, and the Physicians' Desk Reference.

It should be understood that the methods of the invention described herein can be adapted to a high throughput format, thus allowing the examination of a plurality (i.e., 2, 3, 4, or more) of samples and/or biomolecules, which independently can be the same or different, in parallel. A high throughput format provides numerous advantages. For example, a high throughput format allows for the examination/quantitation of two, three, four, etc., different biomolecules, alone or in combination, of a subject. Finally, a high throughput format allows, for example, control samples (positive controls and or negative controls) to be run in parallel with test samples. In addition a high throughput method may allow immunoprecipitation of multiple proteins at the same time using multiple antibodies.

In another aspect, the invention provides a kit for performing the methods of the invention. In one embodiment, a kit is provided for diagnosing and/or monitoring the progression or treatment of a neurological or neurodegenerative disease in a subject. The kit includes one or more labeled moieties (e.g., labeled amino acids) and a means for administering the one or more amino acids to the subject. The kit may further include a means for obtaining a biological sample at regular time intervals from the subject. In certain embodiments, the kit will also include instructions for detecting and determining the ratio of labeled to unlabeled biomolecules of interest over time and for calculating the concentration of the unlabeled biomolecule. In one embodiment, the instructions will disclose methods for comparing the calculated concentration to certain standards and/or controls as disclosed herein.

In another embodiment, the kit of the invention provides a compartmentalized carrier including one or more containers containing the labeled moiety and the various means for performing the methods of the invention.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Metabolic Labeling of Tau

This example demonstrates that cells producing Tau will metabolically incorporate a stable isotope labeled amino acid into newly synthesized Tau.

We grew cells in normal media until almost confluent. We then added fresh media and let the cells condition the media for 24 hours. Then we spiked in ¹³C₆ leucine into the media at a final ratio of 1:1 for unlabeled to labeled leucine. Media was collected at various time points after addition of ¹³C₆ leucine and Tau was isolated from the media and analyzed using our standard IP/MS protocol. Ratio of labeled to unlabeled Tau was plotted against time.

This illustrates the feasibility of metabolically labeling Tau using ¹³C₆ leucine. This is critical for showing that SILK-Tau is feasible as well as showing that SISAQ quantitation peptides can be made in cells.

### Example 2

### Quantitation of Tau by SISAQ

¹³C₆ leucine labeled Tau was used as Quantitation Standard and spiked into a standard curve of samples containing concentrations of Tau ranging from 5 ng/mL to 51 pg/mL. In addition the Quantitation Standard was spiked into CSF from two different individuals. Tau was isolated from the samples using immunoprecipitation and then digested with Trypsin and analyzed by mass spectrometry. The ratio of unlabeled Tau to Quantitation Standard was calculated for all samples and a standard curve generated. The standard curve was linear in the range tested (5 ng/mL to 51 pg/mL) and was used to calculate the concentration of Tau in the CSF samples. The concentration of Tau in the CSF samples was around 1.2 ng/mL and the CV on triplicate measures of Tau concentration was 4% for one CSF sample and 7% for the other CSF sample.

This illustrates the feasibility of using stable isotope labeled Tau as a quantitation standard and relating the ratio of unlabeled to labeled Tau to a standard curve to allow for measurement of concentrations of Tau in unknown samples.

Although the invention has been described with reference to the above example, it will be understood that modifications and variations are encompassed within the spirit and scope of the invention. Accordingly, the invention is limited only by the following claims.

1. A method of calculating the concentration of a biomolecule in a subject comprising:
(a) contacting a sample from the subject with a Quantitation Standard, wherein the Quantitation Standard comprises a known concentration of a labeled biomolecule of interest;
(b) isolating the biomolecule of interest from the sample, wherein (a) and (b) can occur in reverse order;
(c) determining a ratio of labeled to unlabeled biomolecules in the sample; and
(d) calculating the concentration of the unlabeled biomolecule in the sample.

2. The method of claim 1, wherein calculating the concentration of the unlabeled biomolecule comprises multiplying the known concentration of the Quantitation Standard with the ratio of labeled to unlabeled biomolecules in the sample.

3. The method of claim 1, further comprising normalizing the calculated concentration to a standard curve, wherein the standard curve is generated by determining two or more ratios of unlabeled to Quantitation Standard, wherein the concentration of the unlabeled biomolecule is known.

4. The method of claim 1, wherein the Quantitation Standard comprises one or more labeled moieties.

5. The method of claim 4, wherein the one or more labeled moieties comprise a non-radioactive isotope that is selected from the group consisting of ²H, ¹³C, ¹⁵N, ¹⁷O, ¹⁸O, ³³S, ³⁴S, and ³⁶S.

6. The method of claim 1, wherein the biomolecule is selected from the group consisting of peptides, lipids, nucleic acids, and carbohydrates.

7. The method of claim 6, wherein the biomolecule is a protein that is synthesized in the central nervous system (CNS).

8. (canceled)

9. The method of claim 4, wherein the labeled moiety is a labeled amino acid.

10-30. (canceled)

31. The method of claim 1, further comprising comparing the concentration of the unlabeled biomolecule of interest to the concentration of the same biomolecule in a corresponding normal sample, to the concentration of the same biomolecule in a subject of known neurological or neurodegenerative disease state, to the concentration of the same biomolecule from the same subject determined at an earlier time, or any combination thereof.

32. The method of claim 1, wherein the neurological or neurodegenerative disease is selected from the group consisting of Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases, Lewy Body Disease, Pick's Disease, motor neuron diseases, restless leg syndrome, seizure disorders, tremors, depression, mania, anxiety disorders, brain trauma or injury, narcolepsy, sleep disorders, autism, normal pressure hydrocephalus, pain disorders or syndromes, migraines, headaches, spinocerebellar disorders, muscular dystrophies, myasthenia gravis, retinal degeneration and Amyotrophic Lateral Sclerosis.

33. An in vivo method of quantifying the concentration of one or more biomolecules in a subject comprising:
(a) administering-one or more labeled amino acids to the subject, wherein the labeled amino acids incorporate into a biomolecule of interest in the subject;
(b) obtaining a sample of biological fluid or tissue from the subject, wherein the sample comprises a labeled biomolecule fraction and an unlabeled biomolecule fraction;
(c) contacting the sample with a Quantitation Standard, wherein the Quantitation Standard comprises a known concentration of a biomolecule labeled with a moiety that has a molecular weight that differs from the one or more labeled amino acids administered to the subject;
(d) determining a ratio of labeled to unlabeled biomolecules in the sample, a ratio of labeled biomolecule to the Quantitation Standard, and a ratio of unlabeled biomolecule to the Quantitation Standard; and
(e) calculating the concentration of the unlabeled biomolecule and the concentration of the labeled biomolecule in the sample.

34. The method of claim 33, wherein calculating the concentration of the unlabeled biomolecule comprises multiplying the concentration of the Quantitation Standard with the determined ratio of unlabeled biomolecule to the Quantitation Standard.

35. The method of claim 33, wherein calculating the concentration of the labeled biomolecule comprises multiplying the concentration of the Quantitation Standard with the determined ratio of labeled biomolecule to the Quantitation Standard.

36. The method of claim 33, further comprising normalizing the calculated concentration of unlabeled biomolecule to an unlabeled standard curve, and normalizing the calculated concentration of labeled biomolecule to a labeled standard curve, wherein each of the standard curves is generated by determining two or more ratios of unlabeled or labeled biomolecules to Quantitation Standard, wherein the concentration of unlabeled or labeled biomolecule is known.

37. The method of claim 33, wherein the labeled amino acid and the Quantitation Standard are independently labeled with a non-radioactive isotope selected from the group consisting of ²H, ¹³C_{,} ¹⁵N, ¹⁷O, ¹⁸O, ³³S, ³⁴S, and ³⁶S.

38. The method of claim 37, wherein the labeled amino acid is an essential or nonessential amino acid.

39-49. (canceled)

50. The method of claim 33, wherein the biomolecule is selected from the group consisting of a peptide, a lipid, a nucleic acid, and a carbohydrate.

51. The method of claim 50, wherein the biomolecule is a peptide that is synthesized in the central nervous system (CNS).

52-61. (canceled)

62. The method of claim 33, wherein known concentrations of one or more labeled amino acids are administered to the subject and the concentrations of two or more biomolecules are calculated.

63-65. (canceled)

66. The method of claim 33, wherein the sample is selected from the group consisting of cerebral spinal fluid (CSF), blood, plasma, urine, saliva, and tears.

67-76. (canceled)

77. The method of claim 33, further comprising comparing the concentration of the unlabeled biomolecule of interest to the concentration of the same biomolecule in a corresponding normal sample, to the concentration of the same biomolecule in a subject of known neurological or neurodegenerative disease state, to the concentration of the same biomolecule from the same subject determined at an earlier time, or any combination thereof.

78. The method of claim 77, wherein the neurological or neurodegenerative disease is selected from the group consisting of Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases, Lewy Body Disease, Pick's Disease, motor neuron diseases, restless leg syndrome, seizure disorders, tremors, depression, mania, anxiety disorders, brain trauma or injury, narcolepsy, sleep disorders, autism, normal pressure hydrocephalus, disorders or syndromes, migraines, headaches, spinocerebellar disorders, muscular dystrophies, myasthenia gravis, retinal degeneration and Amyotrophic Lateral Sclerosis.

79. A kit for diagnosing or monitoring the progression or treatment of a neurological or neurodegenerative disease in a subject, the kit comprising:
(a) one or more labeled amino acids;
(b) means for administering the one or more labeled amino acids to the subject, whereby the labeled amino acids are capable of incorporating into and labeling a biomolecule of interest in the subject;
(c) means for obtaining a biological sample at regular time intervals from the subject, wherein the sample comprises a labeled biomolecule fraction and an unlabeled biomolecule fraction;
(d) instructions for detecting and determining the ratio of labeled to unlabeled biomolecules of interest over time and for calculating the concentration of the unlabeled biomolecule in the sample, whereby the concentration of unlabeled biomolecule may be compared to the concentration of the same biomolecule in a corresponding normal sample, to the concentration of the same biomolecule in a subject of known neurological or neurodegenerative disease state, to the concentration of the same biomolecule from the same subject determined at an earlier time, or any combination thereof.

80. The kit of claim 79, wherein the labeled amino acid is an essential or nonessential amino acid.

81. (canceled)

82. The kit of claim 79, wherein the labeled amino acid comprises a non-radioactive atom.

83. The kit of claim 82, wherein the non-radioactive atom is selected from the group consisting of ²H, ¹³C, ¹⁵N, ¹⁷O, ¹⁸O, ³³S, ³⁴S, and ³⁶S.

84. The kit of claim 79, wherein the biomolecule is selected from the group consisting of a peptide, a lipid, a nucleic acid, and a carbohydrate.

85. The kit of claim 84, wherein the biomolecule is a peptide that is synthesized in the central nervous system (CNS).

86-89. (canceled)

90. The kit of claim 79, wherein the neurological or neurodegenerative disease is selected from the group consisting of Alzheimer's Disease, Parkinson's Disease, stroke, frontal temporal dementias (FTDs), Huntington's Disease, progressive supranuclear palsy (PSP), corticobasal degeneration (CBD), aging-related disorders and dementias, Multiple Sclerosis, Prion Diseases, Lewy Body Disease, Pick's Disease, motor neuron diseases, restless leg syndrome, seizure disorders, tremors, depression, mania, anxiety disorders, brain trauma or injury, narcolepsy, sleep disorders, autism, normal pressure hydrocephalus, pain disorders or syndromes, migraines, headaches, spinocerebellar disorders, muscular dystrophies, myasthenia gravis, retinal degeneration and Amyotrophic Lateral Sclerosis.

91-92. (canceled)


SEQUENCE LISTING (12 sequences)

SEQ ID NO: 1 - protein, 16 aa, Homo sapiens
IGSLDNITHVPGGGNK

SEQ ID NO: 2 - protein, 15 aa, Homo sapiens
SGYSSPGSPGTPGSR

SEQ ID NO: 3 - protein, 30 aa, Homo sapiens
STPTAEAEEAGIGDTPSLEDEAAGHVTQAR

SEQ ID NO: 4 - protein, 23 aa, Homo sapiens
KESPLQTPTEDGSEEPGSETSDA

SEQ ID NO: 5 - protein, 17 aa, Homo sapiens
KIGSLDNITHVPGGGNK

SEQ ID NO: 6 - protein, 23 aa, Homo sapiens
KESPLQTPTEDGSEEPGSETSDA

SEQ ID NO: 7 - protein, 30 aa, Homo sapiens
KIATPRGAAPPGQKGQANATRIPAKTPPAP

SEQ ID NO: 8 - protein, 30 aa, Homo sapiens
KSTPTAEAEEAGIGDTPSLEDEAAGHVTQA

SEQ ID NO: 9 - protein, 34 aa, Homo sapiens
KSTPTAEAEEAGIGDTPSLEDEAAGHVTQARMVS

SEQ ID NO: 10 - protein, 22 aa, Homo sapiens
KIGSTENLKHQPGGGKVQIINK

SEQ ID NO: 11 - protein, 36 aa, Homo sapiens
KSTPTAEAEEAGIGDTPSLEDEAAGHVTQARMVSKS

SEQ ID NO: 12 - protein, 34 aa, Homo sapiens
QDQEGDTDAGLKESPLQTPTEDGSEEPGSETSDA